(19) United States
(12) Patent Application Publication
(10) Pub. No.: US 2010/0167271 A1
(43) Pub. Date: Jul. 1, 2010

(54) METHOD FOR SCREENING BLOOD USING A PRESERVATIVE THAT MAY BE IN A SUBSTANTIALLY SOLID STATE FORM

(75) Inventor: Wayne L. Ryan, Omaha, NE (US)

Correspondence Address: DOBRUSIN & THENNISCH PC, 29 W LAWRENCE ST, SUITE 210, PONTIAC, MI, 48342, US

(73) Assignee: Streck, Inc., LaVista, NE (US)

(21) Appl. No.: 12/646,204

(22) Filed: Dec. 23, 2009

Related U.S. Application Data
(60) Provisional application No. 61/141,329, filed on Dec. 30, 2008.
(60) Provisional application No. 61/237,943, filed on Aug. 28, 2009.

(51) Int. Cl. C12Q 1/70; C12Q 1/02; C12Q 1/48; C12M 1/34

(52) U.S. Cl. 435/5; 435/29; 435/15; 435/287.1

(57) ABSTRACT

Methods and devices useful for screening a blood product for a transfusion, pursuant to which leukocytes in drawn whole blood are contacted with a formaldehyde releaser screening preservative so that the presence of any residual leukocytes can be screened. A substantially solid state form preservative for one or more blood components (e.g., leukocytes) and use thereof is also described.

### CLAIM OF PRIORITY

This application claims the benefit of the filing dates of U.S. Provisional Application Ser. Nos. 61/141,329, filed on Dec. 30, 2008 and 61/237,943, filed on Aug. 28, 2009, the entirety of the contents of these application being hereby expressly incorporated by reference.

### FIELD OF THE INVENTION

This invention relates to preservation of biological samples (e.g., blood components) for screening, and more particularly to a method for treating leukocyte-reduced blood to facilitate screening of residual blood components (e.g., residual white blood cell components) in transfusion blood products.

### BACKGROUND OF THE INVENTION

It continues to be popular to treat or manage diseases or disorders through the use of blood product transfusions. One approach that increasingly has gained popularity is the employment of leukocyte-reduced (also referred to as “leuko-reduced”) blood products. Leukoreduction involves the removal nearly all (e.g., >99.9%) of the leukocytes from a quantity of blood in an effort to reduce the possibility of disease transmittal and other associated immune system risks. Conventional removal of the leukocytes has been done by washing techniques, by filtration, or by apheresis. Considerable efforts have been directed toward development of improved leuko-reduction technologies. One concern that has faced developers and prospective users of the technologies is quality assurance. For instance, it is important in either or both of the handling and preparation of leuko-reduced blood products that ample quality control measures are invoked to help assure that transfusion-related adverse reactions are minimized, that disease transmission is reduced, or both. The emphasis on quality control is illustrated, for example, in a January 2001 Publication from the U.S. Department of Health and Human Services Center for Biologics Evaluation and Research, entitled Draft Guidance for Industry—Pre-Storage Leukocyte Reduction of Whole Blood and Blood Components Intended for Transfusion found at www.fda.qov/bioloqicsbloodvaccines/quidancecompliancerequlatorvinformation/quidanc es/blood/ucm076769.htm, incorporated by reference.

The increasingly widespread adoption of leuko-reduction practices has placed an even greater emphasis on the need to monitor efficacy of such practices. One approach has been to monitor residual white blood cell components in leuko-reduced blood products, whether to detect the presence or absence of residual components, the quantity of residual cell components, and/or even the possibility of a disease condition detectable from analysis of the residual white blood cell components. Some of these monitoring steps can be performed manually. Others may be employed using semi-automated or automated techniques. For example, the use of microscopy, hemocytometry (e.g., using a Nageotte hemocytometer), or both has been employed. One increasingly popular approach has been to employ flow cytometry to assess residual white blood cell components in leukoreduced blood. This is the subject of Palmer et al, “Flow Cytometric Determination of Residual White Blood Cell Levels in Preserved Samples from Leukoreduced Blood Products”, Transfusion, Vol. 48 (January 2008), incorporated by reference.

Quality control of leukoreduction procedures involves the screening of a number of randomly selected filtered blood units and thereafter performing a residual white blood cell count. This screening is generally performed within 24 hours of leukoreduction so that the screening results provide an accurate count of the white blood cell component concentration of a leukoreduced blood pack. The acceptable level of residual white blood cells within a leukoreduced blood product is generally less than about 1×10⁶ white blood cells per pack or about 3.3 white blood cells per μl. Thus at such low concentrations, traditional cell preservation methods may not sufficiently preserve the white blood cell components as necessary for accurate screening. Even if only a few white blood cells are not effectively preserved, inaccurate screening results may allow a blood pack to be distributed when in fact the amount of white blood cell components exceeds the acceptable level.

Palmer et al identify that a particular concern facing the implementation of any assay method for monitoring residual white blood cell components is the need to assure that samples analyzed retain their initial residual white blood cell counts. Palmer et al address the use of a Pallfix preservative (which is believed to be a liquid paraformaldehyde solution), by Pall Corp., particularly when the time between sample removal and testing exceeds 1 to 2 days.

Examples of efforts in the art toward assuring quality in large scale blood collection, such as for obtaining blood for transfusions are illustrated in published U.S. Patent Application No. 20070251337 and PCT Application No. WO2006/023725 (PCT/US2005/029559), incorporated by reference. Efforts at the stabilization or decontamination of blood components for transfusion applications are illustrated, for example, in U.S. Pat. Nos. 7,358,039; 6,190,609 and 6,030,767. See also, U.S. Pat. Nos. 6,527,957 and 6,884,573. U.S. Pat. Nos. 5,858,699; 6,197,540; 6,197,539; and 6,579,672 address treating leukocytes with a transition metal solution, which is believed to be commercially marketed under the name TRANSFIX, by Cytomark Limited.

Treatment of cell components with certain formaldehyde releaser materials are disclosed in U.S. Patent Application Nos. 20040137417; 20020119503; 20020086346; see also, U.S. Pat. Nos. 5,196,182; 5,250,438; 5,260,048; 5,459,073; 5,460,797; 5,811,099; 5,849,517; and 6,337,189 all of which are hereby incorporated by reference.

Other patents of potential interest to the present invention may include U.S. Pat. Nos. 7,044,941; 6,994,790; and 5,783,093, all incorporated by reference.

Until the present invention, there has been a need for an efficient, reliable and simple approach to the assurance of the integrity of results from screening of blood products for transfusion. There also has been the need for a preservative composition that makes handling and sample preparation and preservation more convenient. The present invention addresses one or more of the above needs by providing an improved method for treating and/or screening of biological samples, such as blood products, and particularly leuko-reduced blood products.

### SUMMARY OF THE INVENTION

By way of summary, the present invention meets some or all of the above needs by providing a first aspect of the invention directed generally to a biological sample preservative, as described herein, which is provided in a substantially solid state form that includes an agent in a sufficient concentration so that upon contact with the sample (e.g., sample of blood) the screening preservative composition will disperse in the sample, and substantially preserve components, such as white blood cell components, in the sample. The agent may be selected from the group consisting of: diazolidinyl urea, imidazolidinyl urea, dimethoylol-5,5-dimethylhydantoin, dimethylol urea, 2-bromo-2.-nitropropane-1,3-diol, an oxazolidine, sodium hydroxymethyl glycinate, 5-hydroxymethoxymethyl-1-1aza-3,7-dioxabicyclo [3.3.0]octane, 5-hydroxymethyl-1-1-aza-3,7dioxabicyclo [3.3.0]octane, 5-hydroxypoly[methyleneoxy]methyl-1-1aza-3,7dioxabicyclo [3.3.0]octane, quaternary adamantine and any combination thereof.

One specific example of screening preservative composition has a composition of about 5 to about 20% (e.g., about 12.5%) by weight of the agent (e.g., preferably diazolidinyl urea, imidazolidinyl urea, or a combination thereof); and about 0.001 to about 1 mg % (e.g., about 0.1 mg %) of an optional dye; and, optionally, about 0.5 to about 1.2% (e.g., about 0.85%) by weight of an anti-coagulant (e.g., K₃EDTA). This aspect further contemplates that the screening preservative be capable of preserving white blood cell components at extremely low concentrations. For example, the screening preservative may preserve white blood cell components in concentrations of less than about 20 white blood cells per μl. The screening preservative may preserve white blood cell components in concentrations of less than about 10 white blood cells per μl. The screening preservative may preserve white blood cell components in concentrations of less than about 4 white blood cells per μl, or even less than about 3 white blood cells per μl.

This aspect contemplates that the substantially solid state preservative may be substantially free of any crystallinity (e.g., as confirmed by x-ray diffraction analysis) and/or may have a viscosity that is sufficiently high that when placed in a storage device, the substantially solid state preservative will remain substantially immobile (e.g., in a substantially fixed position) in the storage device, regardless of the angle of inclination of the storage device, but will readily disperse within a sample (e.g., a blood product sample) for preserving components (e.g., residual white blood cell components and/or other blood components) within the sample. The invention also contemplates methods of making a substantially solid state preservative that includes removing liquid (e.g., via one or more drying steps) to form the substantially solid state preservative, such as is taught herein.

A second aspect of the invention contemplates a method for screening a blood product for a transfusion, comprising the steps of contacting a leuko-reduced drawn blood sample with a screening preservative composition that is one or both of (i) in a substantially solid state form, (ii) includes a formaldehyde releaser agent selected from the group consisting of diazolidinyl urea, imidazolidinyl urea, dimethoylol-5,5-dimethylhydantoin, dimethylol urea, 2-bromo-2.-nitropropane-1,3-diol, an oxazolidine, sodium hydroxymethyl glycinate, 5-hydroxymethoxymethyl-1-1aza-3,7-dioxabicyclo [3.3.0]octane, 5-hydroxymethyl-1-1-aza-3,7dioxabicyclo [3.3.0]octane, 5-hydroxypoly[methyleneoxy]methyl-1-1aza-3,7dioxabicyclo [3.3.0]octane, quaternary adamantine and any combination thereof; and optionally, screening any residual leukocytes prior to a blood transfusion.

This aspect may be characterized further by one or any combination of the following features: (i) the formaldehyde releaser agent consists essentially of a formaldehyde releaser and is substantially free of any separately added aldehyde cross-linker at the time of contacting, (ii) the contacting is performed in the absence of applied or energized radiation, or both (i) and (ii). The screening preservative composition may include diazolidinyl urea, imidazolidinyl urea, or a combination thereof as the formaldehyde releaser agent; the screening step is performed substantially at the time when the blood is drawn, within 48 hours after any leukoreduction is performed, at least 48 hours after any leukoreduction is performed, or any combination thereof; the screening preservative composition is employed in an amount less than about 10 μl; the leuko-reduced blood sample contains white blood cells in a concentration of less than about 10 white blood cells per μl prior to contact with the preservative composition and maintains substantially the same white blood cell concentration at the time of screening the blood sample.

Prior to and during the contacting step the screening preservative composition may be substantially free of any crystals (e.g., as verified by x-ray diffraction); prior to and during the contacting step the screening preservative composition has a form (e.g., it is substantially hardened or has a relatively high viscosity) that renders it substantially immobile (e.g., it is substantially resistant to flow for a period of time of at least 24 hours when maintained at a temperature of at least about 60° C.). Prior to and during the contacting step the amount of the screening preservative composition may be less than about 50 μl. Prior to and during the contacting step the screening preservative composition may have a composition of about 0.5 to about 1.2% by weight of K₃EDTA; about 5 to about 20% by weight of the agent; and about 0.001 to about 1 mg % of a dye.

The contacting step may occur in a vial having a volume of less than about 5 ml. The contacting step may occur in a vial made of glass. The screening may include passing a portion of the blood through an automated instrument selected from a hematology analyzer, a flow cytometer or a combination thereof. The screening step may include one or any combination of a step of counting residual white blood cells, immunophenotyping residual white blood cells, microscopically inspecting residual white blood cells, analyzing membrane permeability of residual white blood cells, or detecting the presence of bacteria. The screening may include analyzing for the presence of alanine aminotransferase (ALT), one or any combination of infectious diseases selected from Hepatitis (B and/or C) virus, Human Immunodeficiency (HIV1 and/or 2), Human T-Lymphotropic Virus (HTLV-I and/or HTLV-II), or Syphilis, West Nile Virus, or any combination thereof.

The method may include a separating step that includes filtering to remove leukocytes. The separating step may include removing leukocytes at bedside, in a laboratory, or a combination of both. The screening step may be performed at least 72 hours after the contacting step. The contacting step may be performed in a receptacle having a volume less than about 1 ml, in a sampling chamber provided on a blood bag, or both. The viscosity of the screening preservative composition may be sufficiently high that it does not exhibit any visibly detectable (as seen by the naked eye) flow when held at room temperature on an incline of at least about 45° for at least one hour.

The present invention further contemplates a kit used for performing the method of any of foregoing recitations, such as a kit including at least two or any combination of a closure piercing device, a blood pack, a tube, a needle, reagents, a warming device, a refrigeration device, a label, protective gloves, a needle guard, a filter, a catheter, a tube holder, a sample storage device holder, a clip, a clamp, a tube sealer, a tube welder, or one or more assays.

In another aspect of the invention, there is contemplated a device for screening blood for white blood cell components comprising a receptacle that receives a sample of blood and that is substantially transparent over at least a portion of its area; and a screening preservative composition contacting the receptacle and being visible through the substantially transparent window, the screening preservative composition having a form so that it remains substantially fixed in a single location prior to receiving the sample of blood, but which is of sufficient concentration so that upon contact with the sample of blood the screening preservative composition will disperse in the sample, and substantially preserve white blood cell components in the sample.

This aspect may be further characterized by one or any combination of the following features. The receptacle may have a substantially constant dimension cross section along its length. The receptacle may be substantially transparent and may define a window that spans over at least 50% of the total surface area of the receptacle. The screening preservative composition in the receptacle may have a viscosity that renders it substantially immobile (e.g., resistant to flow for a period of time of at least 24 hours when maintained at a temperature of at least about 60° C.). The receptacle may contain less than about 20 μl of the screening preservative composition. The screening preservative composition may include a colorant. The receptacle may have a ratio of height to width ranging from about 1.5:1 to about 2:1. The receptacle may include (i) a substantially round mouth opening at one of its ends having a diameter of about 5 to about 10 mm (e.g., about 8 mm), (ii) a base having a wall that projects generally convexly outward toward the opening, or both (i) and (ii). The receptacle may receive a volume of about 300 to about 1000 μl (e.g., about 500 μl). Use of the device is free of any step of diluting blood, screening preservative composition, or both, which is introduced into the receptacle.

The device may be made by the steps of forming an aqueous liquid mixture including a formaldehyde releaser and a dye, dispensing the liquid mixture into the receptacle, and removing at least about 70% by volume of any water in the liquid mixture to form a screening preservative composition. The removing step may be performed by drying in a humidity controlled chamber at temperature. Any dispensing step may dispense less than about 250 μl (e.g., about 100 μl) into the receptacle. The screening preservative composition includes an agent selected from the group consisting of selected from the group consisting of: diazolidinyl urea, imidazolidinyl urea, dimethoylol-5,5-dimethylhydantoin, dimethylol urea, 2-bromo-2.-nitropropane-1,3-diol, an oxazolidine, sodium hydroxymethyl glycinate, 5-hydroxymethoxymethyl-1-1aza-3,7-dioxabicyclo [3.3.0]octane, 5-hydroxymethyl-1-1-aza-3,7dioxabicyclo [3.3.0]octane, 5-hydroxypoly[methyleneoxy]methyl-1-1aza-3,7dioxabicyclo [3.3.0]octane, quaternary adamantine and any combination thereof. The screening preservative composition may consist essentially of diazolidinyl urea, imidazolidinyl urea, or a combination thereof. Any removing step may retain at least some volume of water in the liquid mixture. Any removing step may be sufficient so that a colored ring (e.g., a generally annular ring) is visibly defined on a base of the receptacle that substantially circumscribes the base of the receptacle. After any removing step, and prior to introduction of any blood product sample, the screening preservative composition may be present in an amount less than 40 μl (e.g., less than about 25 μl, or even less than about 15 μl).

In yet another aspect of the invention, there is contemplated a preservative composition for a liquid biological sample (e.g., blood) upon which an assay is to be performed that is provided in a substantially solid state form and that includes an agent in a sufficient concentration so that upon contact with the liquid biological sample the screening preservative composition will disperse in the sample, and substantially preserve components (e.g., white blood cells) in the sample. The agent may be selected from the group consisting of selected from the group consisting of: diazolidinyl urea, imidazolidinyl urea, dimethoylol-5,5-dimethylhydantoin, dimethylol urea, 2-bromo-2.-nitropropane-1,3-diol, an oxazolidine, sodium hydroxymethyl glycinate, 5-hydroxymethoxymethyl-1-1aza-3,7-dioxabicyclo [3.3.0]octane, 5-hydroxymethyl-1-1-aza-3,7dioxabicyclo [3.3.0]octane, 5-hydroxypoly[methyleneoxy]methyl-1-1aza-3,7dioxabicyclo [3.3.0]octane, quaternary adamantine and any combination thereof. The screening preservative may preserve white blood cell components present in a leuko-reduced blood sample in concentrations of less than about 10 white blood cells per μl. The screening preservative may preserve white blood cell components in concentrations of less than about 6 white blood cells per μl. The screening preservative may preserve white blood cell components in concentrations of less than about 4 white blood cells per μl, less than about 3 white blood cells per μl, or even less than about 2 white blood cells per μl. The preservative composition may be provided as a particulate, as a relatively thin layer (e.g., thinner than about 1 cm, such as thinner than about 2 mm) coated onto a solid substrate or both.

In another aspect, the present invention is directed generally to a device for screening blood comprising a concave or convex base portion that includes a constant cross-section portion about the longitudinal axis of the device, a variable cross-section portion about the longitudinal axis of the device, and a receiving portion attached to the base portion at an intermediate location.

A further aspect of the invention contemplates a device for screening blood comprising a first end, an intermediate location where the cross section about the longitudinal axis of the device changes from constant to variable, a second end, a non-planar base that extends between the second end and toward or away from the intermediate location, a sectional volume per unit length between the second end and the intermediate location that is less than that between the first end and the intermediate location, wherein a preservative composition is supported between the first end and second end.

In yet another aspect, the invention contemplates a device for screening blood for white blood cell components comprising a receptacle that receives a sample of blood and that is substantially transparent over at least a portion of its area, wherein the receptacle includes a first end, a second end, a base portion located a distance between the first end and second end that divides the receptacle into a receiving portion and an elongated channel portion, wherein the first end and second end are both open. The device further includes a screening preservative composition placed within the receptacle and being visible through the substantially transparent window, the screening preservative composition being in solid form and located in the top portion of the receptacle and being of sufficient concentration so that upon contact with the sample of blood the screening preservative composition will disperse in the sample, and substantially preserve white blood cell components in the sample.

These aspects may be further characterized by one or any combination of the following features. The base portion may include a flat portion. The height of the base portion may be from about 2 mm to about 8 mm. The base portion may end at a second end that is longitudinally spaced from the base by at least 10 mm. The base portion may have the same external dimensions as the receiving portion. The base portion may be tubular and may have one open end. The preservative composition may be molded into a shape of predetermined geometry. The preservative composition may be lyophilized within the base portion so that its resulting shape is analogous to that of the base portion. The device may contain a screening preservative composition including a formaldehyde releaser agent selected from the group consisting of diazolidinyl urea, imidazolidinyl urea, dimethoylol-5,5-dimethylhydantoin, dimethylol urea, 2-bromo-2.-nitropropane-1,3-diol, an oxazolidine, sodium hydroxymethyl glycinate, 5-hydroxymethoxymethyl-1-1aza-3,7-dioxabicyclo [3.3.0]octane, 5-hydroxymethyl-1-1-aza-3,7dioxabicyclo [3.3.0]octane, 5-hydroxypoly[methyleneoxy]methyl-1-1aza-3,7dioxabicyclo [3.3.0]octane, quaternary adamantine and any combination thereof. The screening preservative composition may include diazolidinyl urea, imidazolidinyl urea, or a combination thereof as the formaldehyde releaser agent.

The device may include a plastic tube. The device may include a glass tube. The base portion may include an area that is frustoconical in shape so that a solid form preservative composition is maintained in contact with the base. The second end of the receptacle may receive a label or other identifier for providing information regarding the contents of the receptacle. At least part of the base portion of the receptacle may be frustoconical in shape so that a solid form preservative composition is maintained in contact with the base portion. The solid form preservative composition may be shaped in an analogous shape to the base portion for close fit with the base of the top portion of the receptacle. The second end of the device may terminate at the base portion. The device may have a substantially constant dimension cross section along its length. The device may be substantially transparent and may define a window that spans over at least 50% of the total surface area of the device. The device may have a ratio of height to width ranging from about 8.5:1 to 11:1. The device may receive a volume of about 300 to about 1000 μl (e.g., about 500 μl).

In yet another aspect of the invention, there is contemplated a method for screening a blood product for a transfusion, comprising the steps of contacting a leuko-reduced drawn blood sample with a screening preservative composition that is (i) in a solid state form having no flow capability, and (ii) includes a formaldehyde releaser agent selected from the group consisting of diazolidinyl urea, imidazolidinyl urea, dimethoylol-5,5-dimethylhydantoin, dimethylol urea, 2-bromo-2.-nitropropane-1,3-diol, an oxazolidine, sodium hydroxymethyl glycinate, 5-hydroxymethoxymethyl-1-1aza-3,7-dioxabicyclo [3.3.0]octane, 5-hydroxymethyl-1-1-aza-3,7dioxabicyclo [3.3.0]octane, 5-hydroxypoly[methyleneoxy]methyl-1-1aza-3,7dioxabicyclo [3.3.0]octane, quaternary adamantine and any combination thereof, and optionally, including a step of screening any residual leukocytes prior to a blood transfusion.

This aspect may be further characterized by one or any combination of the following features. The screening preservative composition includes diazolidinyl urea, imidazolidinyl urea, or a combination thereof as the formaldehyde releaser agent. The contacting step may occur in a device having a storage volume of less than about 5 ml. The screening step may be performed substantially at the time when the blood is drawn, within 48 hours after any leukoreduction is performed, at least 48 hours after any leukoreduction is performed, or any combination thereof. The contacting step may occur in a device including a first end, an intermediate location, a second end and a non-planar base. The screening may include passing a portion of the blood through an automated instrument selected from a hematology analyzer, a flow cytometer or a combination thereof, and/or wherein the screening step includes one or any combination of a step of counting residual white blood cells, immunophenotyping residual white blood cells, microscopically inspecting residual white blood cells, analyzing membrane permeability of residual white blood cells, or detecting the presence of bacteria; the screening step is performed at least 72 hours after the contacting step.

In yet another aspect, the present invention contemplates a method of making a device, comprising the steps of forming an aqueous liquid mixture including a formaldehyde releaser and a dye, placing the aqueous liquid mixture into the device, freezing the aqueous liquid mixture, removing at least about 90% by volume of any water in the liquid mixture by drying the frozen aqueous liquid mixture under low pressure conditions to form a screening preservative composition.

This aspect may be further characterized by one or any combination of the following features. The screening preservative composition includes an agent selected from the group consisting of: diazolidinyl urea, imidazolidinyl urea, dimethoylol-5,5-dimethylhydantoin, dimethylol urea, 2-bromo-2.-nitropropane-1,3-diol, an oxazolidine, sodium hydroxymethyl glycinate, 5-hydroxymethoxymethyl-1-1aza-3,7-dioxabicyclo [3.3.0]octane, 5-hydroxymethyl-1-1-aza-3,7dioxabicyclo [3.3.0]octane, 5-hydroxypoly[methyleneoxy]methyl-1-1aza-3,7dioxabicyclo [3.3.0]octane, quaternary adamantine and any combination thereof. The screening preservative composition may consist essentially of diazolidinyl urea, imidazolidinyl urea, or a combination thereof. After the removing step and prior to introduction of any blood product sample, the screening preservative composition may be present in an amount less than 40 μl (e.g., less than about 25 μl, or even less than about 15 μl). The contacting step may occur in a vial having a storage volume of less than about 5 ml.

### BRIEF DESCRIPTION OF THE DRAWINGS

FIG. 1A shows a perspective view of an illustrative storage device in accordance with the present teachings.

FIG. 1B shows a perspective view of the illustrative storage device of FIG. 1A with a cap in a removed state.

FIG. 1C is a side sectional view of an illustrative storage device in accordance with the present teachings.

FIG. 2 is a plan view of an illustrative system for the collection and storage of blood product in accordance with the present teachings.

FIG. 3 is a perspective view of a possible end cap device for delivering a screening composition to a sample (e.g., in the absence of a step of transferring blood from a sample segment of a tubing to a separate storage device).

FIG. 4 shows a perspective view of an illustrative storage device in accordance with the present teachings.

FIG. 5 shows a cross sectional view of the storage device of FIG. 4.

FIG. 6 shows placement of a screening preservative composition according to the present invention into the storage device of FIG. 4.

FIGS. 7A-7C show a cross sectional view of the storage device including a blood sample and showing the screening preservative composition as it disperses into the blood sample over time.

### DETAILED DESCRIPTION

The present invention is predicated, at least in part, upon the recognition of a need in the art for quality assurance improvements for donor blood, and particularly in the achievement of one or any combination of advantages such as the provision of an elegantly simple approach to the preservation of donor blood product samples that substantially reduces handling error; the provision of a reagent that can be easily employed by clinicians in micro-quantities, with inconsequential risk to the integrity of sampling data; the provision of an easy to manipulate storage device that is pre-loaded with reagent in a manner so that, in the event the storage device breaks or tips over, its reagents are not prone to contaminating an environment; or the provision of a device that enables a sample having low concentrations of white blood cell components to be preserved substantially intact for at least 4 weeks, 8 weeks or longer, optionally without refrigeration.

The present invention is also predicated, at least in part, upon the recognition of a need in the art for a preservative composition that is in a highly viscous or substantially solid state, such that (for example) it can be used effectively as a substantially solid state coating or can be formed into a solid pellet. This would render the preservation composition useful in any of a variety of applications calling for use of a preservative (e.g., cell preservation, tissue preservation, or preservation of other biological matter).

The present invention in its various aspects meets some or all of the foregoing needs. To achieve one or any combination of the various advantages of the present invention, there is employed a particular screening preservative composition. As used throughout the present teachings, the screening preservative composition preferably is substantially non-toxic; is free of separately adding and/or handling of any materially significant concentration (e.g., less than about 1% by weight, more preferably less than about 2000 parts per million, more preferably less than about 1000 parts per million, and still more preferably less than about 500 parts per million) of formaldehyde and/or paraformaldehyde prior to any contact with a blood product sample; has a color visually detectable by a naked eye under ordinary ambient lighting; is substantially free of any crystallinity; has a viscosity that is sufficiently high that when placed in a storage device, the composition will remain substantially immobile (e.g., in a substantially fixed position at ambient temperature in the storage device, regardless of the angle of inclination of the storage device); is in a substantially solid state; will readily disperse within a blood product sample for preserving residual white blood cell components and/or other blood components within the sample; or any combination of the foregoing.

The screening preservative compositions herein are of a type and are used in an amount so that residual white blood cell components are preserved and can be counted after a period (e.g., at least 48 hours, at least 96 hours, at least one week, at least one month, or even at least two months) substantially immediately following i) the draw of blood from a donor; ii) the withdrawal of a sample from a red blood cell pack (e.g., one that has been leukoreduced) for quality assurance testing; iii) the withdrawal of a sample from a platelet pack (e.g., one that has been leukoreduced) for quality assurance testing; iv) a step of leukoreduction; or v) any combination of i) through iv). The screening preservative composition preferably will be such that it will not adversely react with or otherwise impair the efficacy of any agents that are added to the blood product sample (e.g., upon draw from a donor); for example, the screening preservative composition may be one that will be compatible with and/or free of any efficacy reducing interaction, any anti-coagulant, pharmaceutical agent, pH adjuster, or other additive (e.g., one or any combination of sodium citrate, citric acid, dextrose, monobasic sodium phosphate, sodium chloride, mannitol, or adenine). It should be appreciated that, throughout the present teachings, references to “donors” contemplate not only donors of blood where the blood is intended for and used to provide a blood product for transfusion into a patient other than the donor, but also donors of blood where the blood is intended for and used to provide a blood product for subsequent transfusion into the donor.

Preferred screening preservative compositions herein make use of an unexpected characteristic of the compositions that they can be dispensed in micro-quantities of a liquid solution (e.g., in an aqueous solution in an amount in a range of about 300 to about 1000 μl (e.g., about 500 μl)), and thereafter (but prior to any contact with a blood product sample) the liquid of the solution can be removed (e.g., water can be dried) to reduce the volume of the solution, while leaving a sufficiently high concentration of ingredients in the composition so that the composition will readily disperse within a blood product sample for preserving residual white blood cell components and/or other blood components within the sample. Preferred screening preservative compositions herein make use of another unexpected characteristic of the compositions in that upon any liquid removal, the remaining composition (which may be present in the form of a substantially solid and/or substantially immobile coating) can be seen unaided by a human eye within any receptacle into which it is dispensed (e.g., it will have a color visually detectable by a naked eye under ordinary ambient lighting). Preferred screening preservation compositions also may form a highly viscous or substantially solid form, such as a coating film that can bond to a substrate or a solid composition in the form of a pellet that can be easily placed into a receptacle.

In general, the compositions herein may include, consist essentially of, or even consist of an optional colorant and a formaldehyde releaser component (preferably one that is substantially free of any aldehyde cross-linker prior to any contact with a blood product sample; e.g., it has less than about 1% by weight, more preferably less than about 2000 parts per million, more preferably less than about 1000 parts per million, and still more preferably less than about 500 parts per million, of formaldehyde and/or paraformaldehyde). A preferred formaldehyde releaser component may include, consist essentially of or even consist of an agent selected from the group consisting of: diazolidinyl urea, imidazolidinyl urea, dimethoylol-5,5-dimethylhydantoin, dimethylol urea, 2-bromo-2.-nitropropane-1,3-diol, an oxazolidine, sodium hydroxymethyl glycinate, 5-hydroxymethoxymethyl-1-1aza-3,7-dioxabicyclo [3.3.0]octane, 5-hydroxymethyl-1-1-aza-3,7dioxabicyclo [3.3.0]octane, 5-hydroxypoly[methyleneoxy]methyl-1-1aza-3,7dioxabicyclo [3.3.0]octane, quaternary adamantine and any combination thereof. Among the most preferred formaldehyde releaser components are diazolidinyl urea, imidazolidinyl urea or a combination thereof.

When employed, examples of colorants include (without limitation) FD&C Red #3, Red #40, Yellow #5, Yellow #6, Blue #1, Blue #2, and Green #3, or any combination thereof. Preferably, the colorant will be of a type and will be used in an amount so that it does not materially degrade any of the preservative performance obtained by the formaldehyde releaser. The colorant will be of a type and will be used in an amount so that it imparts a visibly detectable contrast with the color of the sample with which it comes into contact. In this way, a step may be performed of ascertaining whether the screening preservative composition has been fully dispersed into the sample, by inspecting a treated sample for the presence of any visible colorant. In the case of a coating, a colorant may be employed in an amount sufficient to visibly distinguish from (e.g., contrast with) the glass or other material of the receptacle (or any other substrate upon which it is coated) so that the presence of the compositions can be verified. A colorant may also be employed in the case of a substantially solid composition placed within the receptacle. A substantially solid composition may also be free of any colorant so long as the solid composition is visible within the receptacle without the aid of a colorant. The relative concentration (in weight) of the colorant and the formaldehyde releaser component preferably will range from about 1 part colorant to about 10 to about 100,000 (e.g., about 100 to about 10,000) parts formaldehyde releaser component.

The preparation of the compositions herein may involve the use of a liquid diluent. One approach may be to disperse colorant and formaldehyde releaser component in deionized and/or distilled water. For example, about 1 part by volume of the formaldehyde releaser component may be mixed with about 1 to about 1000 (e.g., about 10 to about 100) parts by volume of the liquid (e.g., water). Colorant may be mixed with the formaldehyde releaser component prior to during or after the formaldehyde releaser component is mixed with the liquid (e.g., water).

The selection of ingredients for the screening preservative compositions may be such that the resulting composition (in either or both of its diluted state prior to any liquid diluent removal or subsequent to any liquid diluent removal) has a pH in the range of about 5.5 to about 8.5, and more preferably about 6.5 to about 7.5 (e.g., about 7.2). The screening preservative compositions may be hypertonic or hypotonic. For example, the screening preservative compositions may be such that the resulting composition (in either or both of its diluted state prior to any liquid diluent removal or subsequent to any liquid diluent removal) has an osmolarity of at least about 400 milli-osmoles per kilogram, and more preferably at least about 475 milli-osmoles per kilogram (e.g., about 400 to about 600 milli-osmoles per kilogram, or even about 500 milli-osmoles per kilogram).

The screening preservative composition may preserve white blood cell components at very low concentrations as generally found in leuko-reduced blood products. As an example, the concentration of white blood cell components within a leuko-reduced blood pack may be less than about 1×10⁶ white blood cells per unit, or less than about 3.3 white blood cells per μl. The screening preservative may preserve white blood cell components in concentrations of less than about 20 white blood cells per μl. The screening preservative may preserve white blood cell components in concentrations of less than about 10 white blood cells per μl. The screening preservative may preserve white blood cell components in concentrations of less than about 4 white blood cells per μl, or even less than about 3 white blood cells per μl. The screening preservative may be effective so that preservation of at least about 50%, at least about 75%, or even at least about 90% of the white blood cell components in a blood sample is achieved. The screening preservative may effectively preserve 100% of the white blood cell components within a blood sample.

It is possible that the resulting compositions can be employed thereafter in a diluted state (e.g., in the form of an aqueous solution) in accordance with the present teachings. However, one preferred approach includes one or more steps of removing at least a portion of the liquid diluent from the compositions. Effectively, it is possible to thus obtain a concentrate that includes the formaldehyde releaser component. Generally, preservative compositions in a diluted state require a substantial amount of liquid reagent for effective preservation. Alternatively, the concentrated form of the preservative composition may allow for effective preservation with only a small amount of reagent. This small amount of concentrated preservative is surprisingly able to effectively contact and preserve white blood cell components at very low concentrations, as discussed above.

The liquid removal step may include a lyophilization process. The composition in a diluted state may be frozen to below its triple point to ensure proper removal of water during latter drying steps. The frozen diluted composition may then be dried under low pressure conditions and with a specific heat level so that the water moves directly from solid to gas form. Any water that remains after the drying process may be removed via an additional drying step. The resulting lyophilized solid pellet will distribute into solution quickly upon combination with any sample fluids (e.g. blood samples or the like). The solid composition can also be easily pre-loaded into any receptacle or alternatively added after blood draw. The lyophilization process may occur outside of the receptacle with the resulting preservative composition being placed into the receptacle post-lyophilization. The lyophilization process may also occur within the receptacle.

Where the preservative composition is formed into a substantially solid pellet, the pellet may be substantially symmetrical about the x, y, and/or z orthogonal axes. The pellet may also be asymmetrical about one or more of the x, y, and z axes. The pellet may also include a tapered end. The pellet may also be shaped so that it nestingly resides in the receptacle. The pellet may be shaped so that it is held in place via contact with one or more walls of the receptacle. At least one end of the pellet may be generally frustoconical and/or rounded (e.g. hemispherical). The shape of the pellet may also facilitate a desired dissolution rate once the pellet comes into contact with a sample. Where the lyophilization process occurs within the receptacle, the pellet may be formed into a shape that is analogous to that of the base of the receptacle. The pellet may also be formed in a shape analogous to any location within a receptacle where the preservative composition exists prior to the lyophilization process.

Another approach may be to dispense into a receptacle a first volume of a composition that includes, consists essentially of, or even consists of the formaldehyde releaser component, any optional colorant, and a liquid diluent. During dispensing or after, at least a portion of the liquid diluent is removed (e.g., it is preferentially removed while preserving substantially the same amount of the dispensed formaldehyde releaser component and any colorant). This might be performed by a step of drying, and preferably a step of drying in a humidity controlled chamber.

For example, one approach may include a step of placing compositions (e.g., subsequent to dispensing into a storage device or onto some other substrate) including the liquid diluent in a suitable humidity controlled chamber, and maintaining them in the chamber at one or more temperatures and at one or more humidity levels for a period of time sufficient to achieve the desired removal of liquid diluent. By way of illustration, compositions may be placed in a chamber that is held at one or more temperatures of about 15 to about 50° C., and more preferably about 20 to about 30° C. (e.g., about 22° C.). During the liquid removal, the chamber may be operated to have a relative humidity of less than about 40%, more preferably less than about 30% (e.g., about 23%). The temperature and humidity conditions are preferably maintained at approximately atmospheric pressure for a period of at least about 1 hour, more preferably at least about 5 hours, and still more preferably at least about 20 hours (e.g., about 24 hours). It is possible that the conditions may be maintained for a period no greater than about 48 hours, or even no greater than about 36 hours. The chamber optionally may be evacuated. The chamber optionally may employ a blanket of a substantially inert gas.

Liquid removal conditions may preferably be such that they result in removal of at least about 50% by weight, more preferably at least about 75% by weight, and still more preferably at least about 95% by weight of the original amount of the dispensed liquid diluent. Liquid removal conditions may preferably be such that they result in removal of sufficient liquid so that the resulting composition is in the form of a film, gel or other substantially solid or highly viscous layer; for example it may result in a substantially immobile coating (preferably a coating that can be re-dissolved or otherwise dispersed upon contact with a blood product sample).

Thus, liquid removal conditions may preferably be such that they result in a material that upon contact with the sample under consideration (e.g., leuko-reduced blood) the screening preservative composition will disperse in the sample, and substantially preserve components (e.g., residual white blood cell components) in the sample. Liquid removal conditions may be such that they result in a remaining composition that is substantially free of crystallinity; has a viscosity that is sufficiently high that the remaining composition is substantially immobile at ambient temperature (e.g., it does not exhibit any visibly detectable (as seen by the naked eye) flow when held in a storage device at room temperature on an incline of at least about 45° for at least one hour); or both. The screening preservative composition may also be in solid form so that viscosity and mobility are no longer of concern. The solid form may be in the form of a pellet such that a substantial amount of liquid (e.g. substantially all liquid (e.g. greater than 90% by volume or even greater than 95% by volume)) has been removed resulting in a concentrated solid screening preservative composition.

In the case of a more viscous composition, one possible approach to confirm that a composition herein is substantially immobile is to provide a glass capillary tube open at both ends and having a constant inner diameter (e.g., of about 3 mm). A bottom end is capped and/or a base portion is added at some distance between the two ends of the tube. The tube is filled with a screening preservative composition prior to removal of liquid diluent, and the sample is treated to remove the liquid. The resulting level of the remaining screening preservative composition is recorded. The tube is inverted approximately 180°, the bottom end cap is removed, and flow of the remaining sample in the tube is monitored (at room temperature), by comparing the change in position of the level of remaining screening preservative composition due to gravity, over one or more fixed periods of time. A sample that is substantially immobile will exhibit less than about a 50% (more preferably less than about 25%, or even less than about 10%) change in position of its level over a period of at least one hour, and more preferably will exhibit less than about a 50% (more preferably less than about 25%, or even less than about 10%) change in position of its level over a period of at least one week. One highly preferred approach is to remove sufficient liquid so that crystallinity formation (as confirmed by x-ray diffraction analysis) is substantially avoided in the resulting screening preservative composition, and the resulting screening preservative composition exhibits less than about 3% change in position of its level over a period of at least one week.

For example, liquid removal conditions may preferably be such that they result in a remaining composition that has a form (e.g., a substantially solid, or highly viscous form) that renders it substantially immobile and/or resistant to flow for a period of time of at least 24 hours when maintained at a temperature of at least about 60° C. Thus, liquid removal conditions may be such that the remaining composition forms a coating (e.g., a substantially immobile coating film or other layer in direct contact with an inner surface of the storage device, such as a wall and/or base of the receptacle, and/or a membrane or other seal or substrate) or a substantially solid pellet that includes a composition as described herein. For example, the coating may continuously or intermittently cover at least a portion of a surface. The coating may have a thickness less than about 1 mm (e.g., less than about 0.5 mm). The coating may define a ring (e.g., a generally annular ring), a line, a curve, or any combination thereof, which itself may have a width less than about 2 mm (e.g., less than about 1 mm). A pellet in substantially solid form may resist any flow by virtue of its solid nature.

One particular approach herein may be to employ a receptacle that receives a biological sample that is subjected to testing, such as a sample of blood (e.g., leukoreduced blood) and that includes a substantially transparent window over at least a portion of its area. A transparent polymeric or glass vial (optionally including a coating or other suitable substantially transparent containment barrier, e.g., a polymeric containment barrier, such as described in U.S. Pat. No. 7,419,832, incorporated by reference herein for all purposes) may be employed. Thus, the substantially transparent portion (e.g., a window) may encompass substantially the entirety of the receptacle. The receptacle may be equipped with a suitable cap or closure, such as a cap or closure that seals an opening of the receptacle, into which a sample is dispensed.

The receptacle may be formed from a tube including a generally cylindrical wall and having two open ends located on a first end and a second end. The first and second end may be separated by a base portion located at a distance between a first end and a second end that is at least about 25% of the distance between the first end and the second end. The base portion may act to divide the receptacle into a first receiving portion that receives a closure and into which a sample is introduced and a second portion that generally includes an elongated channel. One or more portions of the receptacle may also be contacted by the screening preservative composition. The screening preservative composition is preferably located within the receiving portion so that a sample will contact the screening preservative upon placement into the receiving portion. The screening preservative may be in a viscous form so that it can be applied to the any portion of the cylindrical wall of the receptacle in addition to the base portion. The screening preservative may also be in a substantially solid form so that it forms a pellet that can be placed into the receiving portion. The solid form screening preservative may be placed in contact with the base portion and may or may not be analogous in shape to the base portion.

The elongated channel portion of the receptacle may be placed onto a projection so that the fit is relatively loose in that minimum contact occurs between the elongated channel and the projection. The elongated channel and the projection may also be frictionally or mechanically engaged. The projection may include a gripping portion by which a processing or testing apparatus can move, spin, or otherwise process a sample located within the receptacle. The device may further include an outer wall surface that is sufficiently large so that it can receive a label or other identifying device containing sample information while still allowing a substantial portion of the sample itself to be viewable. The label or other identifying device may be placed onto the receptacle at a location below base so that the receiving portion is not contacted by the label or other identifying device.

The base portion may include one or more walls that are concave or convex in shape. The base portion may also include a portion having a constant cross-section about the longitudinal axis of the receptacle. The base portion may also include a portion having a variable cross-section about the longitudinal axis of the receptacle. The base portion may also be substantially flat. The base portion may be curved so that it is hemispherical or frustoconical in shape.

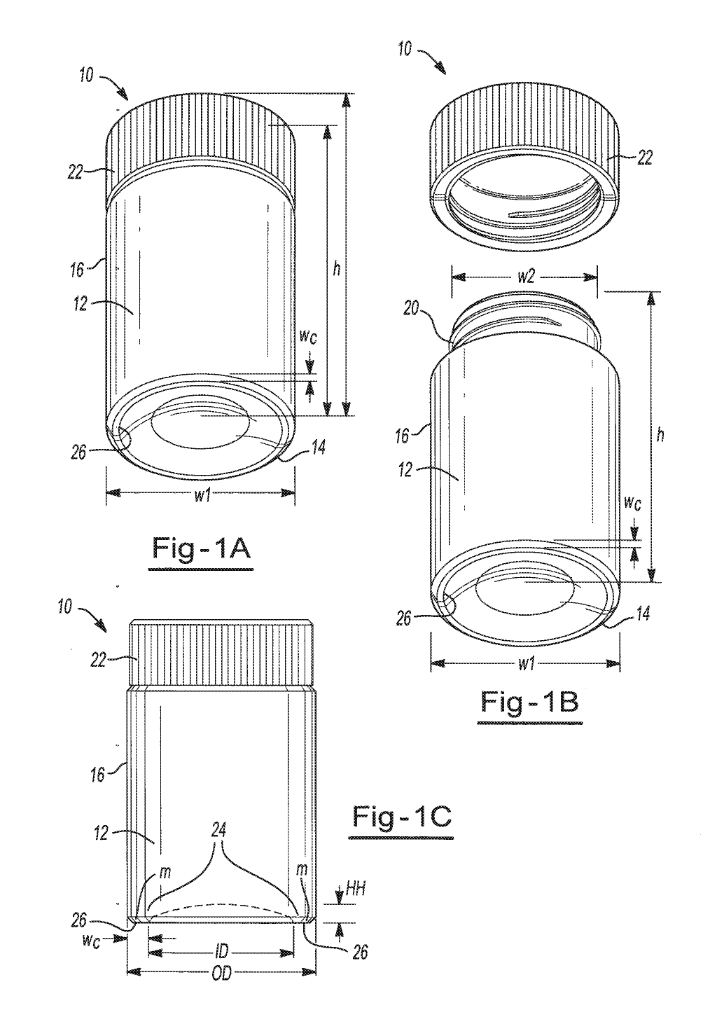

FIGS. 1A-1C illustrate an example of one possible storage device 10. There is shown a receptacle 12 having a base 14, and a vertically projecting sidewall 16 circumscribing the base 14. An opening 18 is defined in an upper portion of the receptacle. The receptacle may have a threaded neck 20, onto which a threaded cap 22 may be screwed. A suitable seal (not shown) may be fitted within the cap or otherwise positioned to form a seal between the cap and a top of the neck. One possible approach may be to form a coating of the compositions herein onto a substrate (e.g., a membrane), which may be used as the seal. The receptacle may include an elongated channel (not shown) that extends downward from the base 14. The elongated channel may also include an additional vertically projecting sidewall that circumscribes the base 14 in an opposing direction to the vertically projecting sidewall 16. The elongated channel may have an open end that allows for a projection to enter the open end and penetrate the elongated channel.

The base of the receptacle may contain a channel or other suitable reservoir that receives the compositions of the invention. For example, as seen in FIG. 1C, one approach may be to employ an upwardly projecting (e.g., an upwardly and convexly projecting) wall, which defines a generally ring-like well 24 at the juncture of the base 14 and sidewall 16. For example the ring-like well may be substantially annular. The base will have a highest height (Hh). Though it is possible that the volume of the compositions that are employed for contacting a sample (e.g., a blood product sample) will be such that is has a meniscus (m) that is vertically higher than Hh, a meniscus, if any, more typically will have a height that is below the height Hh. In accordance with the methods herein it is possible that when the compositions herein are dispensed into the receptacles with the liquid diluent there will be a sufficient volume so that the resulting meniscus is vertically above Hh, but sufficient diluent liquid is removed so that the resulting volume of the composition has a height that is below Hh. The screening preservative compositions may reside in a ring like well to define a ring (e.g., a substantially annular ring) having an inner dimension ID (e.g., inner diameter) and an outer dimension OD (e.g., outer diameter).

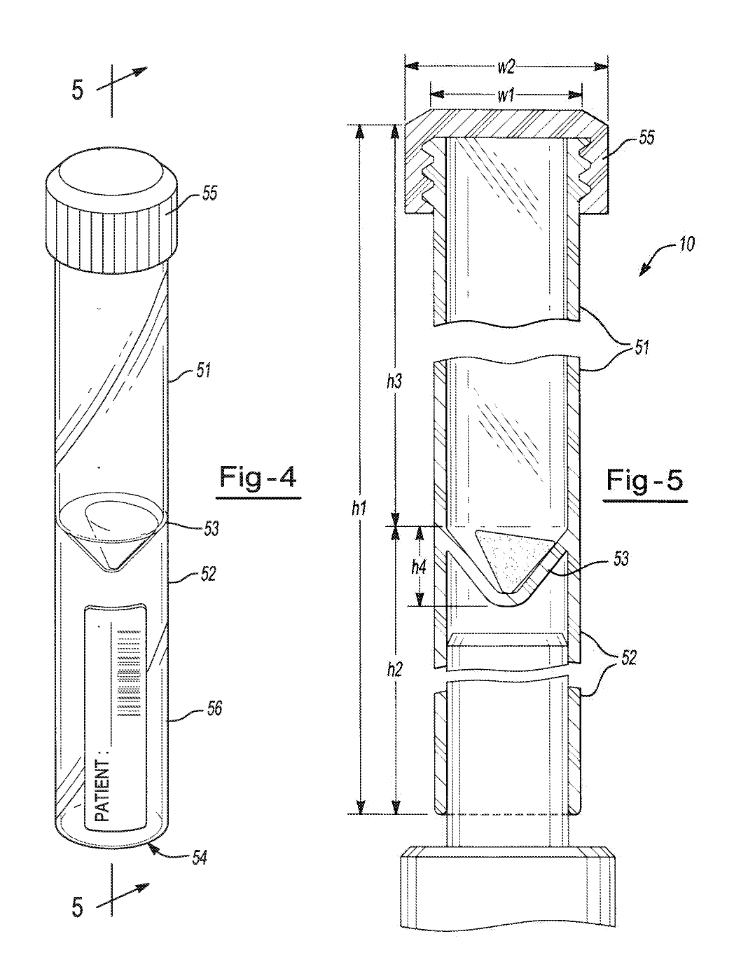

As shown in FIGS. 4 and 5, the receptacle may also include a first end 51 and a second end 52. The first end 51 and second end 52 may be separated by a base 53. The base 53 may be flat, curved, or conical in shape. The second end 52 may be an opened end 54 or a closed end. The receptacle may also include a cap portion 55. The receptacle may also include a label portion 56 such that the label portion does not obfuscate the entirety of the contents within the receptacle. In an illustrative embodiment, the base is generally frustoconical or curved in shape.

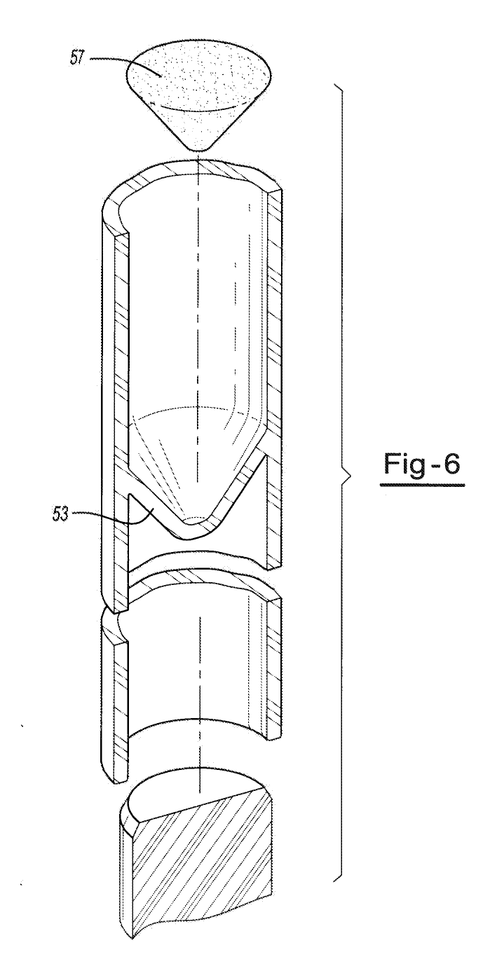

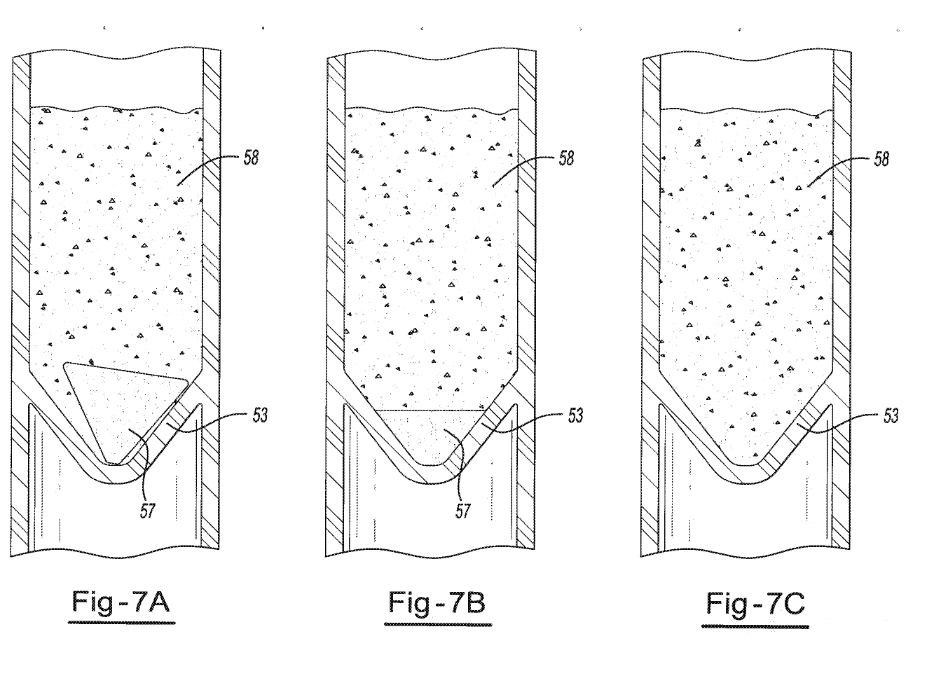

As shown in FIGS. 6, 7A and 7B, the solid form screening preservative composition 57 may be shaped in an analogous frustoconical or curved shape so that the screening preservative 57 is maintained in contact with the base 53. This contact results in reduced movement of the solid form screening preservative thereby reducing the amount of breakage experienced by the screening preservative. The shape of the base may include a varying radius of curvature so that the screening preservative may rest in planar contact with the base, line contact with the base, or point contact with the base depending upon the relative shape and radius of curvature of both the base and the screening preservative. Contact between the solid form screening preservative 57 and the base 53 is easily maintained while the receptacle is maintained in an upright position, but the solid form screening preservative may move freely throughout the receiving portion 51 during any movement of the receptacle. The receptacle and/or screening preservative may further include one or more agents that promote temporary adhesion between the receptacle and the screening preservative. The one or more agents may create a tacky surface on one or more of the receptacle and screening preservative. The one or more agents may also include a mild adhesive that dissolves and/or becomes inactive upon addition of a sample to the receptacle. As shown in FIGS. 7A-7C, the solid form screening preservative will preferably disperse into a blood sample 58 upon contact with the blood sample.

In accordance with the above, it is thus possible that the screening preservative composition will contact the receptacle and be visible through the receptacle (e.g., through any substantially transparent window). As indicated, the screening preservative composition preferably will have a form that is substantially solid or has a sufficiently high viscosity so that it remains substantially solid or substantially fixed in a single location prior to receiving the sample (e.g., leukoreduced blood sample), but will be of sufficient concentration so that upon contact with the sample (e.g., leukoreduced blood) the screening preservative composition will disperse in the sample, and substantially preserve components (e.g., residual white blood cell components) in the sample.

In further reference to FIG. 5, the storage device 10 will preferably have a receptacle that has an overall height (h1) along a longitudinal direction, and a width (w) (i.e. the largest dimension in the transverse direction). For a storage device with a substantially cylindrical receptacle, the width will correspond with the diameter at any given height. As seen, it is possible that there may be two or more widths for the receptacle. For example, there may be a first width (w1) that corresponds substantially with the width of the base, and a second width (w2) that corresponds substantially with the outermost width of the threaded neck or the outermost width of a cap portion. The radius of the cap portion may extend beyond the wall of the receptacle. The ratio of h1:w1 may range from about 0.5:1 to about 15:1, and more preferably about 2:1 to about 11:1. For example, the height may be about 75 to about 125 mm (e.g., about 100 mm), and the first width (w1) may be about 5 to about 25 mm (e.g., about 10 mm). As another example, the height may be about 10 to about 50 mm (e.g., about 30 mm), and the first width (w1) may be about 5 to about 25 mm (e.g., about 15 mm). The width of the neck may be about 3 to about 20 mm (e.g., about 8 mm). The height of the neck portion may range from about 5% to about 60% of the overall height h1, and more preferably will range from about 10% to about 50% of the overall height. It is possible that the width of the neck and the width of the base are approximately the same.

As illustrated in FIG. 5, the receptacle may also include a height h1 that extends the entire portion of the receptacle including any cap portion. The receptacle may also include a height h2 that includes only the elongated channel portion extending downward from the base portion. The receptacle may further include a height h3 that extends from the base portion up to the first end that receives a cap and an additional height h4 that extends from the top of the base to the bottom of the base. The ratio of h3:h2 may range from 1:100 to 3:1. Preferably, the ratio of h3:h2 ranges from 1:4 to 2:1. The height h2 of the elongated channel portion may also be sufficiently long so that it can fit a label containing scanable and/or readable identification information (e.g. a barcode and/or RFID device).

The receptacle may be manufactured by any number of manufacturing methods for plastic or glass materials including but not limited to expansion-resistant glass, polystyrene or polypropylene. The receptacle may be manufactured by welding or a variety of molding techniques (e.g. blow molding, rotational molding, injection molding).

The receptacle may also employ a blood sample identification system such as that disclosed in a co-owned U.S. Provisional Application entitled “Blood Sample Identification System” filed with the USPTO on Aug. 31, 2009, and incorporated by reference herein for all purposes. The identification system includes a handling device for a biological specimen within a receptacle comprising an initially planar substrate including at least one crease that divides the substrate into a handle portion having at least one peripheral edge portion and a receiving portion that includes at least a portion of an aperture. The aperture may include a perimeter that is configured so that it receives a container having a cover that includes a biological specimen for test, and resists pull-through of the container relative to the substrate. The substrate may further include identification information about the sample contained within the receptacle and/or its source.

The storage devices herein may be provided as part of a kit. For example, they may be provided in a kit that includes, consists essentially of, or even consists of at least two or any combination of a closure piercing device, a blood pack, a tube, a needle, reagents, a warming device, a refrigeration device, a label, protective gloves, a needle guard, a filter (e.g., a leukoreduction filter), a catheter, a tube holder, a sample storage device holder, a clip, a clamp, a tube sealer, a tube welder, one or more assays, or any combination thereof.

Blood collection packs may include or be part of a system that includes one or more filters to remove leucocytes and/or thrombocytes from whole blood and a sampling pouch (e.g., for pre-donation screening). The blood collection packs may be part of a single bag system or a multiple bag system (e.g., a system that include one, two, three, four or more bags, each being in fluid communication with at least one other bag, such as via a tubing). Tubing used to fluidly interconnect two or more bags may feature a structure that is substantially resistant to kinks. One or more filters may be included in-line with one or more tubing sections. Bags may be pre-loaded with stabilizer (e.g., for preserving blood product, such as erythrocyte concentrates, contained therein for a predetermined period (e.g., 1 week or longer, 4 weeks or longer, or even 7 weeks or longer)). Bags may be equipped with a venous needle, a suitable connector (e.g., a Luer lock connector) or both.

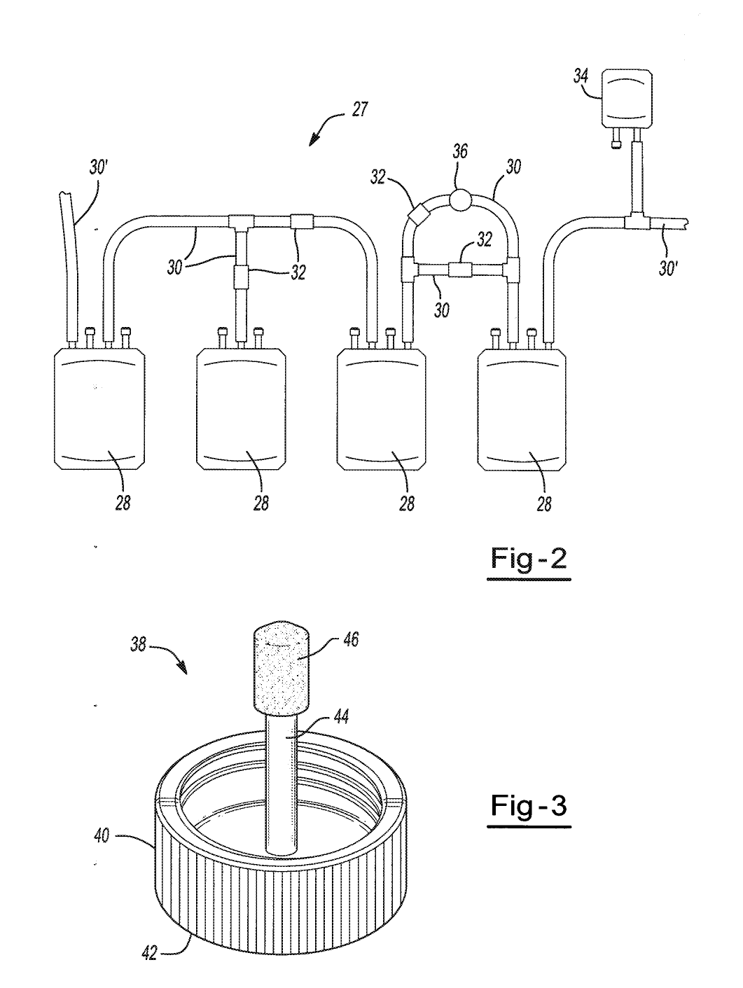

FIG. 2 illustrates an example of one possible blood bag system 27 including a plurality of bags 28 interconnected by tubing 30. The system 27 may include a primary bag, at least one optional satellite bag, and an optional platelet storage bag. The tubing may include one or more in-line device 32 (e.g., valves, connectors, filters or any combination), used in fluidly connecting two or more bags. The system may include an attached sampling pouch 34 and an in-line filter device 36. The invention may include one or more steps of transferring contents between two or more of the primary bag, any satellite bag or the platelet storage bag. Separation of components within the contents may be performed during or after the transferring. One approach is to separate two or more of plasma, platelets and erythrocytes from each other, and thereafter individually storing at least the erythrocytes (and optionally the platelets and/or plasma) in a bag.

Tubing may also be employed for sampling. For example, tubing may extend from a bag (optionally not connected to another bag, such as tubing 30′), from which sampling segments may be made. For example, blood samples may be obtained from tubing connected to a bag into which the original collection is made, into which red-cell components are ultimately contained, or both.

According to one possible system, steps may be performed by which a volume of whole blood in a first bag is centrifuged and then leukocytes, platelets and plasma are transferred to a second bag. The remaining red blood cells may be contacted with a suitable solution (e.g., a solution contained in a third bag of a system) in the first bag. The contents of the first bag may then be filtered into a fourth bag, from which samples are derived for analysis.

Blood collection bags herein may include an integral blood sampling chamber (e.g., a tubing such as tubing 30′) or some other appendage that projects from a pack used to collect blood and that is attached to the pack. In this regard, it is possible that an integrated blood sampling chamber may include the composition of the present invention (e.g., a coating of the composition that will disperse in a blood product sample as taught herein may be included on a substrate in the blood sampling arm). The sampling chamber may be removed from the bag and capped with a sealing device such as described, or otherwise closed (e.g., by welding).

As discussed, the methods herein contemplate a possible step of transferring samples from the system (e.g., transferring samples from one or more of sampling segments of tubing, from a sampling chamber or otherwise) into the storage device herein. It is also possible that a solid pellet of the screening preservative composition of the present invention is placed within the storage device herein or that a coating of the screening preservative composition of the present invention is applied to an inner wall of the tubing or other chamber structure, to a closure or seal for the tubing (e.g., an end cap), or both. For example, one device may include an end cap that has a member that projects from it axially into the tubing. The projecting member may have a coating of the screening preservative composition of the present invention applied to it. It may also be possible that a protective barrier is applied over the screening preservative composition, so that upon initial contact with blood in the tubing, the screening preservative composition does not immediately contact the blood, but after a period of time, the screening preservative composition becomes exposed to the blood. For example, the protective barrier may be a displaceable shield that translates in response to a mechanical and/or electrical actuation; it may be a barrier that erodes; or any combination. FIG. 3 illustrates one example of an end cap sealing device 38 that includes a wall 40 that contacts tubing (over an inner surface, another surface or both of the wall 40) for sealing it, which projects from a base 42. A projection 44 extends from the base, the outer wall or both, and is provided with a coating 46 that includes the screening preservative composition of the present invention.

As seen from the above teachings, it may also be possible to employ a method herein that is free of any step of transferring a blood product sample treated with the screening preservative composition of the present invention from a tubing segment or other blood sampling chamber of a bag to a storage device as described herein. Rather, assays can be performed upon blood obtained directly from a blood bag without transferring to an intermediate storage device. Of course, the methods herein also contemplate a step of transferring a blood product sample to a separate storage device such as shown in the drawings herein (and having the composition of the invention therein), and thereafter testing blood from the storage device. It should be appreciated that the invention herein thus contemplates as storage devices not only the storage devices discussed herein and illustrated with reference to the present drawings, but also blood sampling chambers that may be integrally associated with a blood collection bag.

In use of the compositions, devices and/or methods of the invention, preferably under sterile conditions, blood is drawn from a donor and collected in a blood collection pack. The blood may be leuko-reduced (e.g., within 72 hours of the blood draw). A sample portion of the blood (e.g., the resulting blood product following leuko-reduction) is contacted with the composition herein as taught in the foregoing (e.g., after any step of leukoreduction). The resulting preserved blood is thereafter screened for a disease condition.

Screening of a blood product sample may be for the purpose of identifying the existence of one or more disease conditions. For example, screening may involve performing one or more assays upon a blood product sample for identifying an indicator of HIV-1, HIV-2 (the viruses that cause AIDS), hepatitis B, hepatitis C, human T-cell lymphotropic viruses (HTLV-I and HTLV-II), ALT (the level of a liver enzyme), or syphilis. Screening may include a step of screening for West Nile Virus.

The screening may include a step of testing a blood product sample to identify whether a blood product sample is from a person who currently and/or previously was infected and/or to detect whether the blood product sample is from a person who is a carrier of an infection. The screening may include a step of testing a blood product sample to analyze genetic material associated with a particular disease condition. The screening may include a step of testing a blood product sample to analyze the existence of a disease condition prior to onset of any symptoms of the disease.

Tests performed upon a blood product sample may be performed manually. Tests may be performed by a semi-automated instrument. Tests may be performed by an automated instrument. Tests may be performed proximate the blood draw site (e.g., on the premises of a blood draw site, such as on the premises of a blood donor center). Tests may be performed at a location remote from the blood draw site. For example, a step of transporting a blood product sample to a remote site such as a clinical laboratory (e.g., a remote site that is at least about 1 km from the blood draw site, at least about 25 km from the blood draw site, or even at least about 100 km from the blood draw site) for testing may be employed. During the transporting the blood product sample may be refrigerated (e.g., to a temperature at least 10° C. below ambient temperature, or even at least about 15° C. below ambient temperature). During the transporting step, the blood product sample may be free from any refrigeration, so that it is exposed to ambient conditions.

Prior to, during or subsequent to screening, a step of quarantining a blood product pack may be employed. The quarantining may last until a sample from the blood product pack is tested and shown to be free of infectious agents, or for an additional period of time thereafter. It is also possible that the methods herein will include screening a blood product sample and thereafter issuing a recall of a supply of blood based upon the results of the screening.

Stability is possible for at least 48 hours (more preferably at least 96 hours and still more preferably at least one week) following introduction of a blood product sample into a sample storage device of the present teachings, following blood draw or both; and the methods therefore contemplate analyzing a treated blood product sample at least 48 hours (more preferably at least 96 hours and still more preferably at least one week; and still more preferably at least 4 weeks, and even still more preferably at least 8 weeks) following introduction of the sample into the sample storage device of the present teachings, the blood draw, or both. From the time of introducing the screening preservative compositions herein into a storage device, the compositions should remain stable for a period of at least about 30 days, more preferably at least about 60 days, and still more preferably at least about 90 days.

Though described in connection with a coating on a sample collection container, the compositions herein have broader application. For example, the coating may be formed upon a substrate other than a sample collection container. It may be formed as a particulate or other solid form that is unsupported by any substrate. It may be formed upon a solid substrate. It may be formed upon a porous substrate. It may be formed upon a substrate that is a metal, a plastic, a ceramic or a composition of any of the foregoing. It may be formed upon a relatively rigid substrate (e.g., a substrate capable of supporting its own weight and the weight of the coating without sagging). It may be formed upon a flexible substrate that is not relatively rigid. The coating may have one or more thicknesses. For example, it may have a maximum thickness less that about 8 mm or even less that about 5 mm (e.g., about 2 mm or less).

Though described in connection with an application for screening donor blood, the compositions herein (particularly those in the form of a coating or other substantially solid state) can be employed in methods that include a step of contacting the composition in its substantially solid state with a biological sample to be preserved. It may be employed in place of, or in addition to, the liquid compositions taught for applications described in published U.S. Patent Application Nos. 20040137417; 20020119503; 20020086346; see also, U.S. Pat. Nos. 5,196,182; 5,250,438; 5,260,048; 5,459,073; 5,460,797; 5,811,099; 5,849,517; 6,337,189; all of which are hereby incorporated by reference. The compositions also may be employed to make cellular analogs for use as quality reference controls, as taught in U.S. Pat. No. 6,723,563, incorporated by reference.

As can be appreciated, the screening step may include one or any combination of a step of performing flow cytometry, performing an immunoassay, counting residual white blood cells, immunophenotyping residual white blood cells, microscopically inspecting residual white blood cells, analyzing membrane permeability of residual white blood cells, or detecting the presence of bacteria.

As can be appreciated from the foregoing, the present invention offers many aspects that can be employed to distinguish the methods and compositions herein from existing alternatives. The compositions used for the screening preservative may be: free of photosensitizers; free of photochemicals; free of aldehydes (and particularly are free of any separately added formaldehyde and/or paraformaldehyde (especially during any step of preparing the compositions, prior to contacting the composition with a blood product sample, or both)); free of any transition and/or heavy metals; or any combination thereof. The methods may include a step or irradiating the blood or it may be free of any step of irradiating blood. The method may be free of any step of diluting blood prior to introducing the blood into the receptacle. Specifically, the method may be free of any step of diluting blood, subsequent to its leukoreduction and prior to introducing the blood into the receptacle. The methods herein may include or be free of any step of refrigeration of a sample from the time of blood draw until the time of screening.

### EXAMPLE

A generally cylindrical glass vial having a screw cap, such as the storage device of FIGS. 1A-1C, having a volume of about 1000 μl is filled with about 100 μl of an aqueous stock solution that includes about 0.85% by weight ethylenediaminetetraacetic acid (EDTA), about 12.5% by weight imidazolidinyl urea (IDU), and about 0.1 mg/100 ml FD&C #40 red dye. Water from the stock solution is removed from the vial; specifically, the solution is dried by placing in a controlled humidity chamber for about 24 hours at a temperature of about 22° C. and a relative humidity of about 23%. Upon drying, a visible red ring coats the base of the vial.

The vial is positioned on a holder at an angle of about 45° and held for periods of one hour, 24 hours, 72 hours and one week. In each instance, the resulting coating remains substantially static and free of any flow visibly detected by the naked eye.

Thereafter about 500 μl of leukoreduced blood product sample is introduced into the vial. Within a period of less than about 5 minutes, the screening preservative composition is dispersed into the sample so that no visible evidence of the coating remains.

A specimen of untreated blood from which the blood product sample is taken is immediately analyzed as an untreated control specimen to count residual white blood cell components and to analyze for the presence of HIV-1, HIV-2 (the viruses that cause AIDS), hepatitis B, hepatitis C, human T-cell lymphotropic viruses (HTLV-I and HTLV-II), ALT (the level of a liver enzyme), and syphilis. Treated blood product samples are maintained at room temperature for periods of about 24 hours, 72 hours, one week, four weeks, and eight weeks and are then analyzed for comparison with the untreated control specimen. The results show that the number of residual white blood cell components remains within more than about 90% of the untreated control specimen, and surface antigens are preserved for immunophenotyping. Tests performed manually (using a Nageotte counting chamber, Turk's staining solution and optical microscope) and by flow cytometry (measuring fluorescent bead events in nucleic acid dyed cell components) yield substantially identical results. Similar results are also expected when the concentration of IDU is increased (e.g., in amounts to about 20% by weight), or reduced (e.g., in an amount of about 5% by weight); if another of the agents herein is used in such amounts (e.g., diazolidinyl urea); if EDTA is omitted or varied in amount; if another dye is employed; or any combination thereof.

All patents and printed publications referred to herein are expressly incorporated by reference for all purposes. The use of including or comprising (or their derivatives) herein also contemplates the more limiting terms (and their derivatives) of “consisting essentially of” or “consisting of”. “Blood products” herein contemplate within its scope whole blood, or any product that includes one or more components derived from whole blood. Accordingly, “blood products” contemplates product that include red blood cells, plasma, platelets or any combination thereof. As gleaned from the teachings references to screening of leukocytes contemplates not only screening leukocytes as a whole, but perhaps also screening of any component of a leukocyte, such as cytoplasm, membranes, nuclei, or otherwise. Explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the invention. The scope of the invention should, therefore, be determined not with reference to the above description, but should instead be determined with reference to the appended claims, along with the full scope of equivalents to which such claims are entitled. Other combinations are also possible as will be gleaned from the following claims, which are also hereby incorporated by reference into this written description. Unless otherwise set forth, the order of any processing steps should not be regarded as specifying any particular sequence. Thus, for example, a step of separating leukocytes may occur prior to contact with the preservative, after contact with the preservative, or both.

1. A method for screening a blood product for a transfusion, comprising the steps of:
contacting a leuko-reduced drawn blood sample with a screening preservative composition that is (i) in a substantially solid state form; and (ii) includes a formaldehyde releaser agent selected from the group consisting of diazolidinyl urea, imidazolidinyl urea, dimethoylol-5,5-dimethylhydantoin, dimethylol urea, 2-bromo-2.-nitropropane-1,3-diol, an oxazolidine, sodium hydroxymethyl glycinate, hydroxymethoxymethyl-1-1aza-3,7-dioxabicyclo [3.3.0]octane, 5-hydroxymethyl-1-1-aza-3,7dioxabicyclo [3.3.0]octane, 5-hydroxypoly[methyleneoxy]methyl-1-1aza-3,7dioxabicyclo [3.3.0]octane, quaternary adamantine and any combination thereof; and
optionally, screening any residual leukocytes prior to a blood transfusion.

2. The method of claim 1, wherein (i) the formaldehyde releaser agent consists essentially of a formaldehyde releaser and is substantially free of any separately added aldehyde cross-linker at the time of contacting, (ii) the contacting is performed in the absence of applied or energized radiation, or both (i) and (ii).

3. The method of claim 1, wherein the screening preservative composition includes diazolidinyl urea, imidazolidinyl urea, or a combination thereof as the formaldehyde releaser agent.

4. The method of claim 1, wherein the screening step is performed substantially at the time when the blood is drawn, within 48 hours after any leukoreduction is performed, at least 48 hours after any leukoreduction is performed, or any combination thereof.

5. The method of claim 1, wherein the leuko-reduced blood sample contains white blood cells in a concentration of less than about 10 white blood cells/μl prior to contact with the preservative composition and maintains substantially the same white blood cell concentration at the time of screening the blood sample.

6. The method of claim 1, wherein prior to and during the contacting step the screening preservative composition is substantially free of any crystals.

7. The method of claim 1, wherein prior to the contacting step the screening preservative composition has a viscosity that renders it substantially immobile and resistant to flow for a period of time of at least 24 hours when maintained at a temperature of at least about 60° C.

8. The method of claim 2, wherein prior to and during the contacting step the screening preservative composition has a composition of about 0.5 to about 1.2% by weight of K₃EDTA; about 5 to about 20% by weight of the agent; and about 0.001 to about 1 mg % of a dye.

9. The method of claim 1, wherein the screening includes analyzing for the presence of alanine aminotransferase (ALT), one or any combination of infectious diseases selected from Human T-Lymphotropic Virus (HTLV-I and/or HTLV-II), Syphilis, West Nile Virus, or any combination thereof.

10. The method of claim 4, wherein the screening includes passing a portion of the blood through an automated instrument selected from a hematology analyzer, a flow cytometer or a combination thereof, and/or wherein the screening step includes one or any combination of a step of counting residual white blood cells, immunophenotyping residual white blood cells, microscopically inspecting residual white blood cells, analyzing membrane permeability of residual white blood cells, or detecting the presence of bacteria.

11. The method of claim 1, wherein the screening step is performed at least 72 hours after the contacting step.

12. A device comprising:
a) a receptacle that receives a sample of blood and that is substantially transparent over at least a portion of its area;
b) a screening preservative composition contacting the receptacle and being visible through the substantially transparent window, the screening preservative composition having a viscosity so that it remains substantially fixed in a single location prior to receiving the sample of blood, but which is of sufficient concentration so that upon contact with the sample of blood the screening preservative composition will disperse in the sample, and substantially preserve white blood cell components in the sample.

13. A method of making a device according to claim 12, comprising the steps of:
a) forming an aqueous liquid mixture including a formaldehyde releaser and a dye;
b) dispensing the liquid mixture into the receptacle; and
c) removing at least about 70% by volume of any water in the liquid mixture to form a screening preservative composition.

14. The method of claim 13, wherein a freeze drying process is used to remove any water in the liquid mixture.

15. The method of claim 13, wherein at least 90% by volume of water is removed from the liquid mixture to form the screening preservative.

16. The method of claim 13, wherein the screening preservative composition consists essentially of diazolidinyl urea, imidazolidinyl urea, or a combination thereof.

17. The method of claim 13, wherein the screening preservative composition has a composition of about 0.5 to about 1.2% by weight of K₃EDTA; about 5 to about 20% by weight of the agent; and about 0.001 to about 1 mg % of a dye.

18. The method of claim 13, wherein the removing step is sufficient so that a colored ring (e.g., a generally annular ring) is visibly defined on a base of the receptacle that substantially circumscribes the base of the receptacle.

19. A device for screening blood comprising:
a. a first end;
b. an intermediate location where the cross section about the longitudinal axis of the device changes from constant to variable;
c. a second end;
d. a non-planar base that extends between the second end and toward or away from the intermediate location;
e. a sectional volume per unit length between the second end and the intermediate location that is less than that between the first end and the intermediate location;
wherein a preservative composition is supported between the first end and second end.

20. The device of claim 19, wherein the device has a volume of less than about 5 ml between the first end and the non-planar base.
